(19) United States
(12) Patent Application Publication
(10) Pub. No.: US 2023/0144155 A1
(43) Pub. Date: May 11, 2023

(54) SYRINGE WITH BIASING MEMBER

(71) Applicant: CareFusion 303, Inc., San Diego, CA (US)

(72) Inventors: Janice R. PAK, Centennial, CO (US); Edmond Yu, Chino Hills, CA (US); Hojae Choi, Irvine, CA (US); Lun Zhang, Brea, CA (US); Vincent F. Wang, San Gabriel, CA (US); Wesley Underwood, Anaheim, CA (US)

(21) Appl. No.: 17/980,090

(22) Filed: Nov. 3, 2022

Related U.S. Application Data
(60) Provisional application No. 63/278,440, filed on Nov. 11, 2021.

(51) Int. Cl. A61M 5/20

(52) U.S. Cl. CPC A61M 5/2033; A61M 5/2046; A61M 2005/2086

(57) ABSTRACT

Syringes are described herein. In certain embodiments, a syringe includes a syringe body, a movable plunger, and a biasing member. The syringe body defines a cavity and a port in fluid communication with the cavity. The movable plunger is disposed within the cavity. The plunger and cavity define a volume. The port is in fluid communication with the volume. The biasing member is coupled to the plunger. The biasing member is configured to urge the plunger toward the port to dispense a fluid stored within the volume at a desired rate.

### REFERENCE TO RELATED APPLICATIONS

This application claims the benefit of U.S. Provisional Application No. 63/278,440 filed Nov. 11, 2021, which is hereby incorporated by reference in its entirety.

### FIELD OF THE INVENTION

The present disclosure generally relates to syringes, and, in particular, to syringes with biasing members.

### BACKGROUND

Patients in hospitals often receive medications and medical fluids (e.g., a saline solution or a liquid medication) via a vascular access line using a syringe or an intravenous (“IV”) pump. In some applications, it may be desired to keep the vascular access line open when not in continuous use to reduce catheter occlusions and catheter infections.

### SUMMARY

The disclosed subject matter relates to syringes. In certain embodiments, a syringe includes a syringe body defining a cavity and a port in fluid communication with the cavity; a movable plunger disposed within the cavity, wherein the plunger and cavity define a volume and the port is in fluid communication with the volume; and a biasing member coupled to the plunger, wherein the biasing member is configured to urge the plunger toward the port to dispense a fluid stored within the volume at a desired rate.

In certain embodiments, a method is disclosed and includes releasing an energized biasing member coupled to a plunger; advancing the plunger within a syringe cavity; and dispensing fluid from the syringe cavity at a desired rate for a desired period of time in response to releasing the energized biasing member.

In certain embodiments, a syringe includes a syringe body defining a cavity and a port in fluid communication with the cavity; a movable plunger disposed within the cavity, wherein the plunger and cavity define a volume and the port is in fluid communication with the volume; and a biasing member coupled to the plunger, wherein the biasing member is configured to advance the plunger toward the port.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient receiving an infusion of a medical fluid using a syringe.

FIG. 2 is a cross-sectional view of a syringe assembly in accordance with various aspects of the present disclosure.

FIG. 3 is a cross-sectional view of a syringe assembly in an energized state, in accordance with various aspects of the present disclosure.

FIG. 4 is a cross-sectional view of the syringe assembly of FIG. 3 in a released state.

FIG. 5 is a perspective view of a syringe assembly, in accordance with various aspects of the present disclosure.

FIG. 6 is a cross-sectional view of the syringe assembly of FIG. 5.

FIG. 7 is a cross-sectional view of the syringe assembly of FIG. 5.

FIG. 8 is a cross-sectional view of the syringe assembly of FIG. 5 along section line 8-8.

FIG. 9 is a perspective view of a syringe assembly in accordance with various aspects of the present disclosure.

FIG. 10 is a top view of the syringe assembly of FIG. 9.

FIG. 11 is a cross-sectional view of the syringe assembly of FIG. 9 along section line 11-11.

FIG. 12 is a perspective view of a syringe assembly in accordance with various aspects of the present disclosure.

FIG. 13 is a cross-sectional view of the syringe assembly of FIG. 12.

FIG. 14 is a cross-sectional view of the syringe assembly of FIG. 12 along section line 14-14.

FIG. 15 is a detail view of the syringe assembly of FIG. 14.

FIG. 16 is a perspective view of a syringe assembly in accordance with various aspects of the present disclosure.

FIG. 17 is a cross-sectional view of the syringe assembly of FIG. 16.

### DETAILED DESCRIPTION

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to administration of medical fluid by utilizing the disclosed syringe assemblies, it is to be understood that this description is only an example of usage and does not limit the scope of the claims. Various aspects of the disclosed syringe assemblies may be used in any application where it is desirable to administer the flow of fluid.

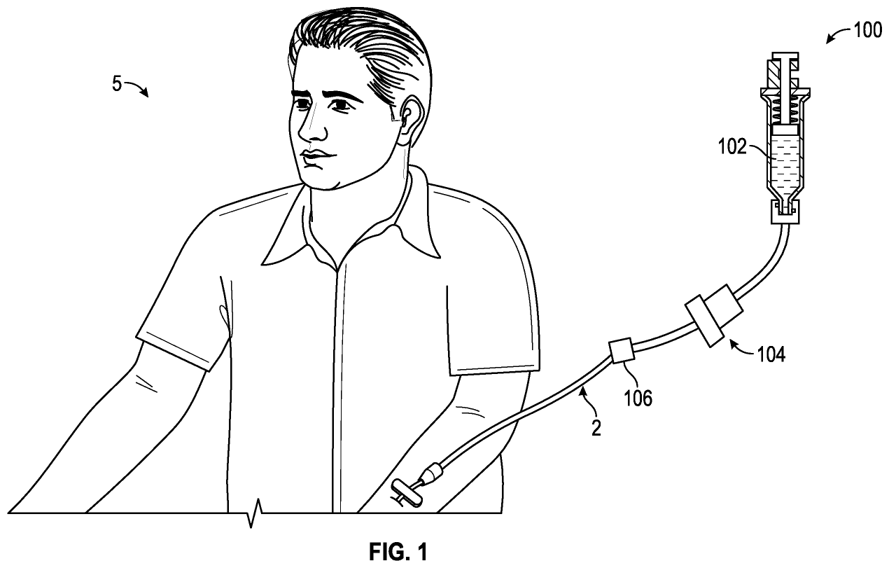

FIG. 1 depicts a patient 5 receiving an infusion of a medical fluid using a syringe assembly 100. In the depicted example, the syringe assembly 100 can deliver a medical fluid from a syringe 102 to the patient 5. In some embodiments, a clinician can actuate the syringe 102 to administer the medical fluid via the vascular access line 2.

During a medical procedure, it may be desirable to provide a controlled amount of fluid to the patient via the vascular access line to keep a vein or vascular access line open when not in continuous use. Advantageously, keeping the vascular access line open can reduce catheter occlusions and catheter infections. In some applications, providing a continuous amount of the fluid to a patient can be therapeutic to patients that are sensitive to fluid buildup. Further, keeping the vascular access line open can increase compliance to vascular access maintenance regulations.

The disclosed syringe assembly can incorporate biasing members to allow for continuous flushing of the vascular access line under controlled pressure using a standalone device. By utilizing the mechanisms described herein, the syringe assembly can provide a standalone device that is portable and requires minimal attention (e.g. every 24 hours).

The disclosed syringe assembly overcomes several challenges discovered with respect to certain approaches to flushing the vascular access line. One challenge with manual flushing of the vascular access line is that manual flushing is time consuming and requires attention of the clinician. Further, intermittent manual flushing may still result in potential occlusions and infections. Another challenge with flushing using a dedicated pump is that a dedicated pump is costly and may be difficult to transport with the patient. Because manual flushing is time consuming, resource intensive and potentially ineffective, and a dedicated pump may be costly and difficult to transport with the patient, it is advantageous to provide a device that provides continuous flushing of the vascular access line in a compact and/or readily portable device. The disclosed syringe assembly is a self-contained assembly that allows for continuous flushing of the vascular access line at a controlled pressure.

Examples of syringe assemblies that allow for continuous flushing of the vascular access line without repeated manual interactions are now described.

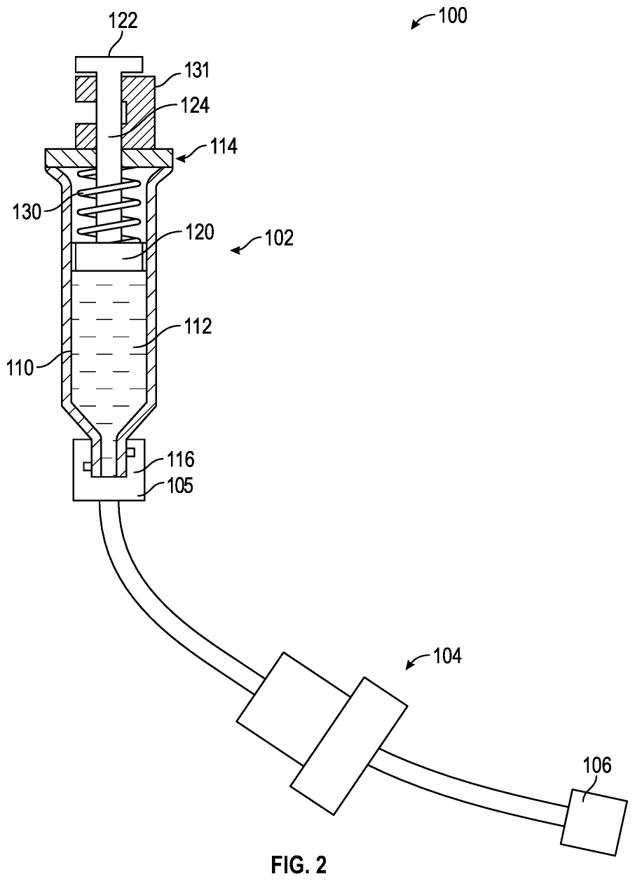

FIG. 2 is a cross-sectional view of a syringe assembly 100 in accordance with various aspects of the present disclosure. In the depicted example, the syringe assembly 100 can deliver fluid to the patient. In some applications, the syringe assembly 100 can continuously deliver fluid to a patient to maintain an unoccupied fluid path in the vascular access line. Advantageously, the syringe 102 can maintain a constant and steady pressure to maintain a small amount of liquid through the patient's vein to maintain an occluded fluid path.

As illustrated, the syringe 102 can store a fluid to be dispensed or administered within a cavity defined by syringe body 110. In some embodiments, the fluid is stored within a syringe volume 112 defined by the syringe body 110 and a movable plunger 120. Fluid can be drawn into the syringe volume 112 through a port 116. Optionally, the syringe volume 112 can be prefilled during assembly. In some embodiments, the fluid is a saline solution and/or another sterile fluid. The syringe 102 can store any suitable amount of fluid. For example, the syringe volume 112 can hold about 1 mL, 2 mL, 5 mL, 10 mL, 20 mL, 30 mL, 40 mL, 50 mL, or 60 mL.

In the depicted example, the plunger 120 can be moved to change or alter the syringe volume 112. In some embodiments, the plunger 120 can move within, or relative to the syringe body 110. The plunger 120 can be movably or resiliently sealed against the walls of the syringe body 110. For example, the plunger 120 can be moved within the syringe body 110 to expand the syringe volume 112. In some applications, the plunger 120 can be withdrawn or moved away from the port 116 to expand the syringe volume 112 and draw in fluid into the syringe volume 112 through the port 116.

To administer or otherwise dispense the fluid within the syringe 102, the plunger 120 can be advanced relative to the syringe body 110 to contract or reduce the syringe volume 112. By advancing the plunger 120 or otherwise reducing the syringe volume 112, the syringe 102 can expel fluid from the syringe volume 112 through the port 116. As illustrated, a clinician can actuate a top portion 122 to advance or retract the plunger 120. The top portion 122 can be coupled to the plunger 120 by a plunger shaft 124.

In the depicted example, the plunger 120 can be automatically advanced relative to the syringe body 110 to allow for continuous fluid delivery through the port 116. Advantageously, by automatically advancing the plunger 120, a vascular access line can be kept open. In some embodiments, a biasing member 130 can be coupled to the plunger 120 to advance the plunger 120 or otherwise urge the plunger 120 toward the port 116. Therefore, the biasing member 130 can allow for continuous fluid delivery through the port 116. The biasing member 130 can be calibrated to apply a desired force or move at a desired velocity to provide a desired flow rate. For example, the biasing member 130 can be calibrated to provide a flow rate of 0.2 mL/hour.

As illustrated, the biasing member 130 can be a spring or any other suitable resilient or energy storing device. In certain applications, an open-coil helical spring can be utilized as a biasing member 130. The biasing member 130 can be energized by moving the plunger 120 to compress the biasing member 130. The biasing member 130 can resist the compressive force to be energized or condensed. The biasing member 130 can advance the plunger 120 when the biasing member 130 is released.

Optionally, the biasing member 130 can be locked or held by a locking member 131 in an energized or compressed state to prevent the inadvertent advancement of the plunger 120 and/or the administration of fluid in the syringe volume 112. In some embodiments, the locking member 131 can prevent movement of the plunger 120. As illustrated, the locking member 131 can be releasably attached to the plunger shaft 124 to prevent movement of the plunger 120. In some embodiments, the locking member 131 can be attached between the top portion 122 of the plunger 120 and the upper portion 114 of the syringe body 110. Optionally, the locking member 131 may couple directly to the biasing member 130 to retain the biasing member 130 in an energized state. The locking member 131 may have a snap fit or interference fit with components of the syringe 102. During operation, the locking member 131 can be removed from the syringe 102 to allow the biasing member 130 to release energy and advance the plunger 120, dispensing fluid from the syringe volume 112.

As illustrated, dispensed or administered fluid from the syringe volume 112 can pass through the port 116 to the patient. The syringe 102 can be in fluid communication with the patient via tubing. In some applications, the tubing can be primed prior to connecting the tubing to the patient's vascular line and/or administering fluid to the patient. As illustrated, the tubing can be connected to the syringe 102 and the patient via luer lock 105 and luer lock 106, respectively.

In some embodiments, a flow controller 104 can control the rate of fluid flow from the syringe 102 to the patient. In particular, the flow controller 104 can start, stop, and adjust the flow from the syringe 102 to the patient. In some embodiments, the flow controller 104 can prevent flow prior to priming and allow flow after the luer lock 106 is connected to the patient. Further, the flow controller 104 can adjust the flow rate of the medical fluid for fluid sensitive patients and/or to adjust the fluid pressure from the syringe 102.

Advantageously, the biasing member 130 and the flow controller 104 can cooperatively generate and control the pressure and fluid flow from the syringe 102 to ensure that the fluid is administered at a constant and steady pressure through a patient's vein to maintain an unoccluded fluid path.

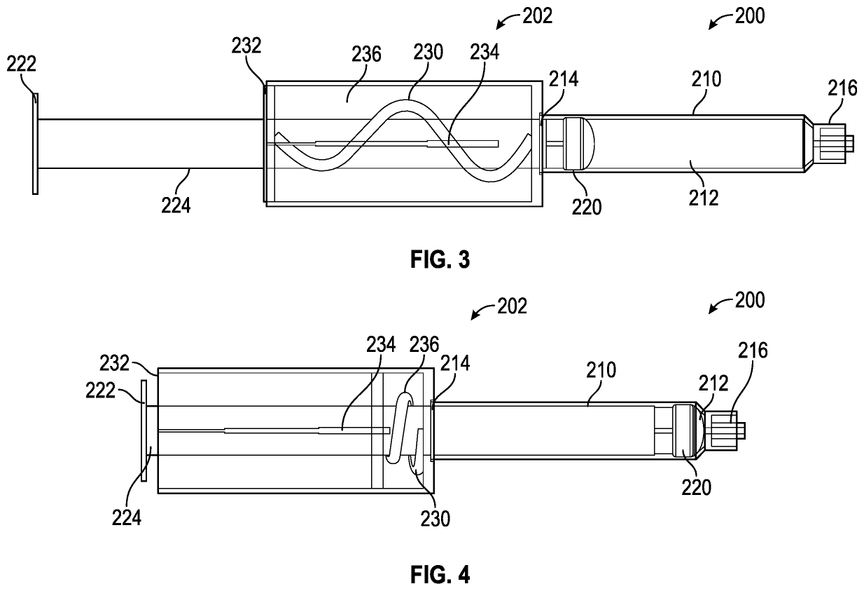

FIG. 3 is a cross-sectional view of a syringe assembly 200 in an energized state, in accordance with various aspects of the present disclosure. FIG. 4 is a cross-sectional view of the syringe assembly 200 of FIG. 3 in a released state. With reference to FIGS. 3 and 4, the syringe assembly 200 utilizes a biasing member 230 and a damper 236 to deliver fluid to the patient at a constant and steady pressure. In some embodiments, the syringe assembly 200 can utilize elements that are similar to the elements of syringe assembly 100. Therefore, for certain elements, similar reference numerals are utilized to refer to similar elements.

Similar to the syringe assembly 100, the plunger 220 can be automatically advanced relative to the syringe body 210 to allow for continuous fluid delivery through the port 216. As illustrated, a biasing member 230 can be coupled to the plunger 220 to advance the plunger 220 or otherwise urge the plunger 220 toward the port 216. The biasing member 230 can be calibrated to apply a desired force or move at a desired velocity to provide a desired flow rate.

As illustrated, the biasing member 230 can be a spring or any other suitable resilient or energy storing device. In certain applications, an open-coil helical spring can be utilized as a biasing member 230. The biasing member 230 can be energized by moving the plunger 220 to extend or tension the biasing member 230. The biasing member 130 can resist the extension force to be energized or tensioned. The biasing member 230 can advance the plunger 220 when the biasing member 230 is released and moves toward a relaxed, condensed state.

In some embodiments, the damper 236 can control the rate of fluid flow from the syringe 202. In particular, the damper 236 can control the velocity of the plunger 220 to allow the plunger 220 to move at a desired rate. In the depicted example, the damper 236 controls the velocity of the damper piston 232, which is coupled to the plunger 220 via a plunger shaft 224. During operation, the flow of damper fluid through the damper 236 controls the velocity of the damper piston 232, and therefore the plunger 220, by exerting force on the damper piston 232. The damper 236 can utilize any suitable fluid. For example, the damper 236 can utilize air to provide damping force.

In some embodiments, the damper 236 can allow for fluid to escape the damper 236 or flow around the damper piston 232 to allow the damper piston 232 to move within the damper 236. As illustrated, the damper 236 can allow for fluid to flow through the damper channel 234 to allow the damper piston 232 to move. Optionally, the size or profile of the damper channel 234 can be modified to adjust the damping force and therefore the velocity of the damper piston 232.

In some embodiments, the damper channel 234 can be varied in size to allow the damper 236 to provide a varied damper response based on the position of the damper piston 232 and/or the plunger 220. As illustrated, an upper portion of the damper channel 234 can have a narrower profile or geometry, allowing for greater damper force and a lower portion of the damper channel 234 can have a wider profile or geometry, allowing for reduced damper force. Advantageously, by varying the geometry of the damper channel 234, the damper 236 can provide a non-linear damper response. A non-linear damper response may be useful for use with biasing members 230 that similarly have a non-linear behavior to provide varying damper force as the biasing force changes. For example, the damper 236 may provide a greater damper force during the initial travel of the plunger 220, when the biasing member 230 provides the greatest biasing force, and less damper force during the end of the travel of the plunger, when the biasing member 230 provides the least biasing force. Advantageously, by correlating the damping force with the biasing force, the damper 236 and the biasing member 230 can cooperatively provide a constant velocity for the damping piston 232 and the plunger 220.

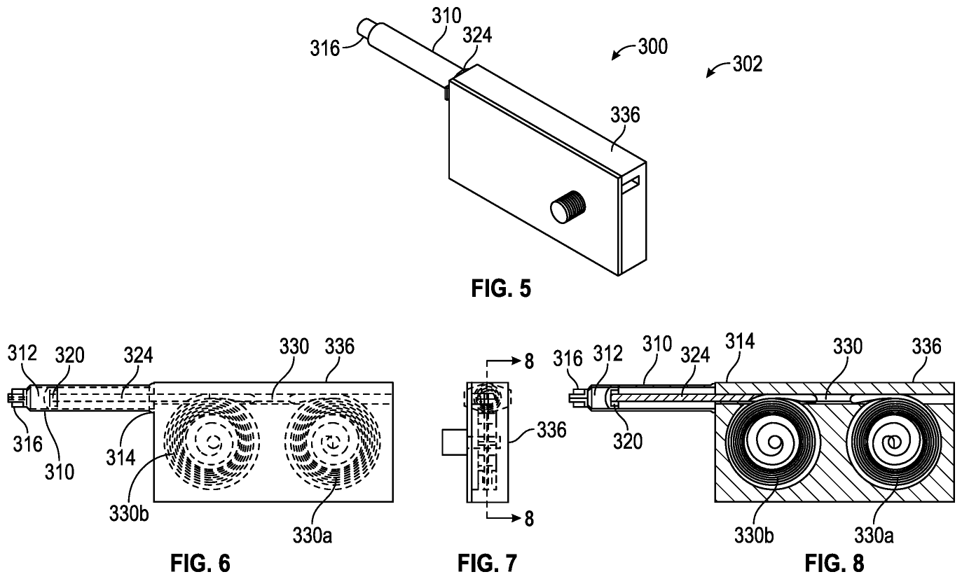

FIG. 5 is a perspective view of a syringe assembly 300, in accordance with various aspects of the present disclosure. FIG. 6 is a cross-sectional view of the syringe assembly 300 of FIG. 5. FIG. 7 is a cross-sectional view of the syringe assembly 300 of FIG. 5. FIG. 8 is a cross-sectional view of the syringe assembly 300 of FIG. 5 along section line 8-8. With respect to FIGS. 5-8, the syringe assembly 300 utilizes a torsional spring 330 to continuously deliver fluid to the patient. Advantageously, the use of a torsional spring 330 can allow for a compact syringe 302 profile. In some embodiments, the syringe assembly 300 can utilize elements that are similar to the elements of syringe assembly 100. Therefore, for certain elements, similar reference numerals are utilized to refer to similar elements.

In the depicted example, the plunger 320 can be automatically advanced relative to the syringe body 310 to allow for continuous fluid delivery through the port 316. Advantageously, by automatically advancing the plunger 320, a vascular access line can be kept open. In some embodiments, the torsional spring 330 can be coupled to the plunger shaft 324 to advance the plunger 320 or otherwise urge the plunger 320 toward the port 316. The torsional spring 330 can be calibrated to apply a desired force or move at a desired velocity to provide a desired flow rate.

As illustrated, the torsional spring 330 can be a coiled or wound spring. The torsional spring 330 can be energized by rotating or coiling the energized portion of the torsional spring 330a. The energized portion of the torsional spring 330a can resist the rotational or torsion to be energized. The plunger 320 can be advanced by the uncoiling of the energized portion of the torsional spring 330a. The deenergized or uncoiled portion of the torsional spring 330b is stored in a reduced energy state in an adjacent coil. In some embodiments, the torsional spring 330a can be energized by retracting the plunger 320.

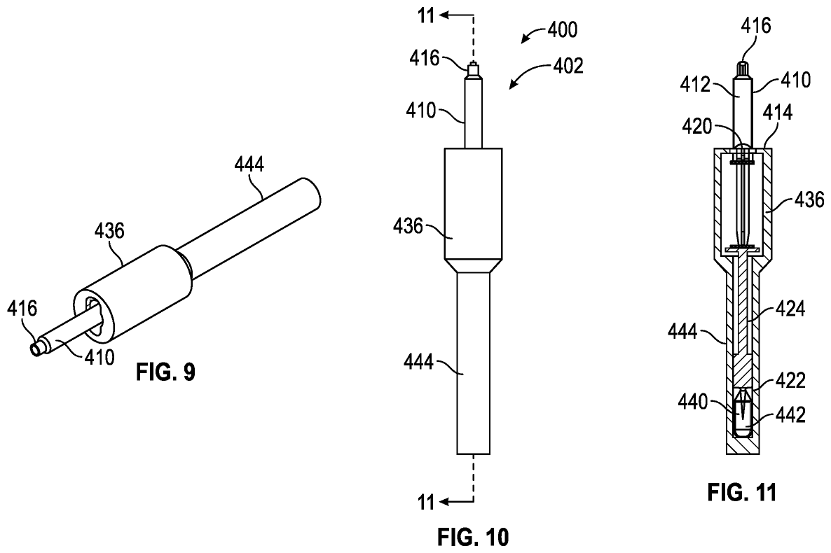

FIG. 9 is a perspective view of a syringe assembly 400 in accordance with various aspects of the present disclosure. FIG. 10 is a top view of the syringe assembly 400 of FIG. 9. FIG. 11 is a cross-sectional view of the syringe assembly 400 of FIG. 9 along section line 11-11. With reference to FIGS. 9-11, the syringe assembly 400 can utilize compressed gas or a chemical reaction to deliver fluid to the patient at a constant and steady pressure. Advantageously, the use of a compressed gas or chemical reaction can allow for a compact syringe 402 profile as well as a syringe 402 that does not need to be energized. In some embodiments, the syringe assembly 400 can utilize elements that are similar to the elements of syringe assembly 100. Therefore, for certain elements, similar reference numerals are utilized to refer to similar elements.

In the depicted example, the plunger 420 can be automatically advanced relative to the syringe body 410 to allow for continuous fluid delivery through the port 416. Advantageously, by automatically advancing the plunger 420, a vascular access line can be kept open. In some embodiments, the plunger shaft 424, and therefore the plunger 420 can be advanced by the expansion of a compressed gas or by the byproduct of a chemical reaction within a gas chamber 442. The gas chamber 442 can be defined by the gas chamber body 444 and the movable plunger shaft 424. As illustrated, the expansion of gasses within the gas chamber 442 can create sufficient force to advance the plunger shaft 424 and therefore the plunger 420 relative to the gas chamber body 444. The expansion of gasses can be controlled to applied a desired force or move the plunger 420 at a desired velocity to provide a desired flow rate.

In some embodiments, compressed gas or chemical reactants can be stored within a capsule 440 within the gas chamber 442. To begin fluid delivery, the capsule 440 can be pierced or punctured to advance the plunger 420. In some embodiments, an upper end 422 of the plunger 420 includes a needle to puncture the capsule 440. Therefore, to initiate administration of fluid, the needle may be advanced into the gas chamber 442 to puncture the capsule 440 and allow the gasses within to expand or react. Optionally, the needle can be advanced into the capsule 440 by moving or actuating the gas chamber body 444. In some embodiments, the depth of the needle and size of the puncture can control the velocity of the plunger 420 and/or the desired flow rate of the syringe 402.

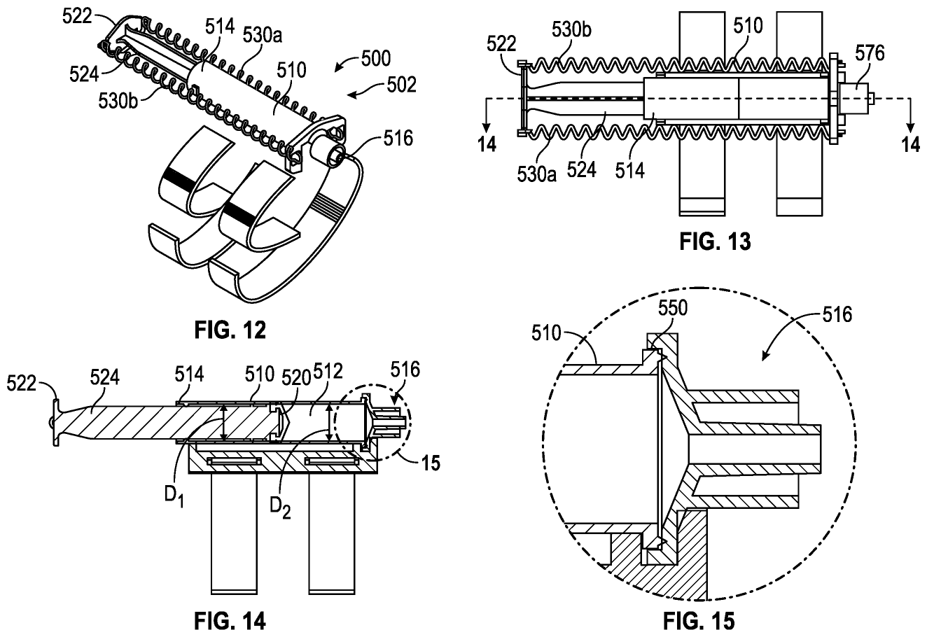

FIG. 12 is a perspective view of a syringe assembly 500 in accordance with various aspects of the present disclosure. FIG. 13 is a cross-sectional view of the syringe assembly 500 of FIG. 12. FIG. 14 is a cross-sectional view of the syringe assembly 500 of FIG. 12 along section line 14-14. FIG. 15 is a detail view of the syringe assembly 500 of FIG. 14. With reference to FIGS. 12-15, the syringe assembly 500 utilizes biasing members 530a and 530b and a tapered syringe body 510 to deliver fluid to the patient at a constant and steady pressure. In some embodiments, the syringe assembly 500 can utilize elements that are similar to the elements of syringe assembly 100. Therefore, for certain elements, similar reference numerals are utilized to refer to similar elements.

Similar to the syringe assembly 100, the plunger 520 can be automatically advanced relative to the syringe body 510 to allow for continuous fluid delivery through the port 516. As illustrated, a biasing members 530a and 530b can be coupled to the plunger 520 to advance the plunger 520 or otherwise urge the plunger 520 toward the port 516. The biasing members 530a and 530b can be calibrated to apply a desired force or move at a desired velocity to provide a desired flow rate.

As illustrated, the biasing members 530a and 530b can be a spring or any other suitable resilient or energy storing device. In certain applications, an open-coil helical springs can be utilized as a biasing members 530a and 530b. The biasing members 530a and 530b can be disposed along the sides of the syringe body 510. The biasing members 530a and 530b can be energized by moving the plunger 520 to extend or tension the biasing members 530a and 530b. The biasing members 530a and 530b can resist the extension force to be energized or tensioned. The biasing members 530a and 530b can advance the plunger 520 when the biasing members 530a and 530b is released and moves toward a relaxed, condensed state.

In some embodiments, the geometry of the syringe body 510 can be utilized to control the rate of fluid flow from the syringe 502. In particular, the diameter of the walls of the syringe body 510 can be adjusted or modified to control the velocity of the plunger 520 to allow the plunger 520 to move at a desired rate. For example, the diameter D1 of the syringe body 510 can be selected to impart a drag force on the plunger 520 as the plunger 520 moves relative to the syringe body 510 to control the velocity of the plunger 520. As illustrated, the geometry of the syringe body 510 can be selected to provide a drag force while still permitting motion of the plunger 520 relative to the syringe body 510.

In some embodiments, the diameter of the syringe body 510 can be varied in size along various portions of the syringe body 510 to allow the plunger 520 to experience a varied drag force based on the position of the plunger 520 relative to the syringe body 510. As illustrated, an upper portion of the syringe body 510 can have a smaller diameter D1, allowing for greater drag force and a lower portion of the syringe body 510 can have a larger diameter D2 allowing for less drag force. Advantageously, by varying the geometry of the syringe body 510, the syringe body 510 can provide a non-linear drag force profile. A non-linear drag force profile may be useful for use with biasing members 530a and 530b that similarly have a non-linear behavior to provide varying drag force as the biasing force changes. For example, the syringe body 510 may provide a greater drag force during the initial travel of the plunger 520, when the biasing members 530a and 530b provide the greatest biasing force, and less drag force during the end of the travel of the plunger 520, when the biasing members 530a and 530b provides the least biasing force. Advantageously, by correlating the drag force with the biasing force, the syringe body 510 and the biasing members 530a and 530b can cooperatively provide a constant velocity for the plunger 520.

With reference to FIG. 15, in some embodiments, the port 516 can be affixed or coupled to the lower end 550 of the syringe body 510. The port 516 may be affixed to the lower end 550 via welding, adhesive, or any other suitable attachment.

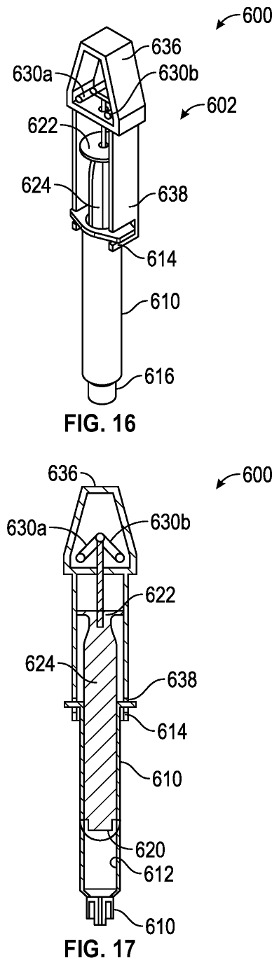

FIG. 16 is a perspective view of a syringe assembly 600 in accordance with various aspects of the present disclosure. FIG. 17 is a cross-sectional view of the syringe assembly 600 of FIG. 16. With reference to FIGS. 16 and 17, the syringe assembly 500 utilizes biasing members 630a and 630b to deliver fluid to the patient at a constant and steady pressure while maintaining a compact syringe 602 profile. In some embodiments, the syringe assembly 600 can utilize elements that are similar to the elements of syringe assembly 100. Therefore, for certain elements, similar reference numerals are utilized to refer to similar elements.

Similar to the syringe assembly 100, the plunger 620 can be automatically advanced relative to the syringe body 610 to allow for continuous fluid delivery through the port 616. As illustrated, a biasing members 630a and 630b can be coupled to the plunger 620 to advance the plunger 620 or otherwise urge the plunger 620 toward the port 616. As illustrated, each of the biasing members 630a and 630b can be coupled to an upper end of the plunger shaft 624. Optionally, as shown, a first end of each of the biasing members 630a and 630b can be coupled to the upper end of the plunger shaft 624 and a second end of the biasing members 630a and 630b can be coupled to a housing 636. As shown, each of the second ends of the biasing members 630a and 630b can be spaced apart in an “arrow” arrangement. Advantageously, this arrangement allows for the biasing members 630a and 630b to be placed in a compact arrangement while allowing for sufficient force to be applied on the plunger shaft 624. The biasing members 630a and 630b can be calibrated to apply a desired force or move at a desired velocity to provide a desired flow rate.

As illustrated, the biasing members 630a and 630b can be a spring or any other suitable resilient or energy storing device. In certain applications, an open-coil helical springs can be utilized as a biasing members 630a and 630b. The biasing members 630a and 630b can be energized by moving the plunger 620 to extend or tension the biasing members 630a and 630b. The biasing members 630a and 630b can resist the extension force to be energized or tensioned. The biasing members 630a and 630b can advance the plunger 620 when the biasing members 630a and 630b is released and moves toward a relaxed, condensed state.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A syringe, comprising: a syringe body defining a cavity and a port in fluid communication with the cavity; a movable plunger disposed within the cavity, wherein the plunger and cavity define a volume and the port is in fluid communication with the volume; and a biasing member coupled to the plunger, wherein the biasing member is configured to urge the plunger toward the port to dispense a fluid stored within the volume at a desired rate.

Clause 2. The syringe of Clause 1, wherein the biasing member comprises a spring.

Clause 3. The syringe of Clause 2, wherein the spring comprises a helical spring.

Clause 4. The syringe of Clause 2, wherein the spring comprises a torsional spring.

Clause 5. The syringe of Clause 1, wherein the biasing member comprises a plurality of springs.

Clause 6. The syringe of Clause 5, wherein a first end of each of the plurality of springs is coupled to the plunger.

Clause 7. The syringe of Clause 1, wherein the biasing member comprises a pressurized gas chamber.

Clause 8. The syringe of Clause 7, further comprising a puncturing member, wherein the puncturing member is configured to pierce the pressurized gas chamber to allow expansion of a pressurized gas.

Clause 9. The syringe of Clause 1, further comprising a damper coupled to the plunger, wherein the damper is configured to control a velocity of the plunger relative to the syringe body.

Clause 10. The syringe of Clause 9, wherein the damper comprises a damper volume and a damper channel in fluid communication with the damper volume, wherein the damper channel permits flow from the damper volume to the environment.

Clause 11. The syringe of Clause 10, wherein the damper channel permits a first damper flow rate at a first plunger position and a second damper flow rate at a second plunger position, wherein the second damper flow rate is greater than the first damper flow rate.

Clause 12. The syringe of Clause 9, wherein the damper comprises a cavity wall and the cavity wall has a first diameter that applies a first damping force at a first plunger position and a second diameter that applied a second damping force at a second plunger position, wherein the first damping force is greater than the second damping force.

Clause 13. The syringe of Clause 1, further comprising a flow controller in fluid communication with the port, wherein the flow controller restricts flow of the fluid at the desired rate.

Clause 14. The syringe of Clause 1, further comprising a locking member releasably attached to the plunger and the syringe body, wherein the locking member is configured to prevent movement of the plunger relative to the syringe body.

Clause 15. A method comprising: releasing an energized biasing member coupled to a plunger; advancing the plunger within a syringe cavity; and dispensing fluid from the syringe cavity at a desired rate for a desired period of time in response to releasing the energized biasing member.

Clause 16. The method of Clause 15, wherein the desired period of time is greater than 12 hours.

Clause 17. The method of Clause 15, wherein the desired rate is about 0.2 mL per hour.

Clause 18. The method of Clause 15, further comprising: controlling the advancing of the plunger via a damping member.

Clause 19. The method of Clause 15, further comprising: removing a locking member to release the energized biasing member.

Clause 20. A syringe, comprising: a syringe body defining a cavity and a port in fluid communication with the cavity; a movable plunger disposed within the cavity, wherein the plunger and cavity define a volume and the port is in fluid communication with the volume; and a biasing member coupled to the plunger, wherein the biasing member is configured to advance the plunger toward the port.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A syringe, comprising:
a syringe body defining a cavity and a port in fluid communication with the cavity;
a movable plunger disposed within the cavity, wherein the plunger and cavity define a volume and the port is in fluid communication with the volume; and
a biasing member coupled to the plunger, wherein the biasing member is configured to urge the plunger toward the port to dispense a fluid stored within the volume at a desired rate.

2. The syringe of claim 1, wherein the biasing member comprises a spring.

3. The syringe of claim 2, wherein the spring comprises a helical spring.

4. The syringe of claim 2, wherein the spring comprises a torsional spring.

5. The syringe of claim 1, wherein the biasing member comprises a plurality of springs.

6. The syringe of claim 5, wherein a first end of each of the plurality of springs is coupled to the plunger.

7. The syringe of claim 1, wherein the biasing member comprises a pressurized gas chamber.

8. The syringe of claim 7, further comprising a puncturing member, wherein the puncturing member is configured to pierce the pressurized gas chamber to allow expansion of a pressurized gas.

9. The syringe of claim 1, further comprising a damper coupled to the plunger, wherein the damper is configured to control a velocity of the plunger relative to the syringe body.

10. The syringe of claim 9, wherein the damper comprises a damper volume and a damper channel in fluid communication with the damper volume, wherein the damper channel permits flow from the damper volume to the environment.

11. The syringe of claim 10, wherein the damper channel permits a first damper flow rate at a first plunger position and a second damper flow rate at a second plunger position, wherein the second damper flow rate is greater than the first damper flow rate.

12. The syringe of claim 9, wherein the damper comprises a cavity wall and the cavity wall has a first diameter that applies a first damping force at a first plunger position and a second diameter that applied a second damping force at a second plunger position, wherein the first damping force is greater than the second damping force.

13. The syringe of claim 1, further comprising a flow controller in fluid communication with the port, wherein the flow controller restricts flow of the fluid at the desired rate.

14. The syringe of claim 1, further comprising a locking member releasably attached to the plunger and the syringe body, wherein the locking member is configured to prevent movement of the plunger relative to the syringe body.

15. A method comprising:
releasing an energized biasing member coupled to a plunger;
advancing the plunger within a syringe cavity; and
dispensing fluid from the syringe cavity at a desired rate for a desired period of time in response to releasing the energized biasing member.

16. The method of claim 15, wherein the desired period of time is greater than 12 hours.

17. The method of claim 15, wherein the desired rate is about 0.2 mL per hour.

18. The method of claim 15, further comprising:
controlling the advancing of the plunger via a damping member.

19. The method of claim 15, further comprising:
removing a locking member to release the energized biasing member.

20. A syringe, comprising:
a syringe body defining a cavity and a port in fluid communication with the cavity;
a movable plunger disposed within the cavity, wherein the plunger and cavity define a volume and the port is in fluid communication with the volume; and
a biasing member coupled to the plunger, wherein the biasing member is configured to advance the plunger toward the port.
